(19) United States
(12) Patent Application Publication
(10) Pub. No.: US 2005/0208003 A1
(43) Pub. Date: Sep. 22, 2005

(54) Topical formulation including stabilized water-soluble and oil-soluble compositions

(76) Inventor: Shyam K. Gupta, Scottsdale, AZ (US)

Correspondence Address: TOD R NISSLE, PO BOX 55630, PHOENIX, AZ, 85078, US

(21) Appl. No.: 11/126,013

(22) Filed: May 10, 2005

Related U.S. Application Data
(63) Continuation-in-part of application No. 10/307,240, filed on Nov. 29, 2002, now abandoned.

(51) Int. Cl. A61K 7/135; A61K 9/64

(52) U.S. Cl. 424/62

(57) ABSTRACT

A topical formulation includes a water-soluble ingredient and an oil-soluble ingredient and includes a combination of water, a rheology modifier, and a water miscible organic solvent to stabilize the composition.

This is a continuation-in-part of U.S. patent application Ser. No. 10/307,240, filed Nov. 29, 2002.

### This invention relates to compositions that include both water-soluble and oil-soluble components.

More particularly, the invention relates to compositions that stabilize simultaneously water-soluble and oil-soluble ingredients.

In a further respect, the invention relates to ascorbic acid compositions that stabilize unusually large concentrations of vitamin C and vitamin E.

In another respect, the invention relates to a stabilized ascorbic acid—vitamin E composition that utilizes water in stabilizing ascorbic acid and vitamin E.

In still a further respect, the invention relates to a composition that utilizes a rheology modifier to absorb water to stabilize water-soluble and oil-soluble components in the composition.

In still another respect, the invention relates to a composition that utilizes a water miscible organic solvent to stabilize water-soluble and oil-soluble components in the composition.

The following terms used herein have the meanings set forth below.
Absorb. To swallow up, suck up by capillary, osmotic, solvent or chemical action.
Adsorb. To take up by physical or chemical forces of molecules of gas, of dissolved substances, or of liquids or by the surfaces of solids or liquids with which they are in contact.
Colloid. A substance that (a) apparently is dissolved, but diffuses slowly through a membrane; or (b) is in a fine state of subdivision so particles are too small to be visible in an ordinary optical microscope and do not settle, or settle slowly.
Dispersion. An emulsion or suspension. Comprises the dispersed substance and the medium it is dispersed in.
Dissolve. To intermix homogeneously to cause to pass into solution.
Emulsion. Intimate mixture of two incompletely miscible liquids so one liquid is dispersed with or without an emulsifier into the other liquid.
Flow. The quantity of a material that flows during a specified period of time under certain conditions, i.e., the motion characteristics of a fluid.
Hydrophilic. Strong affinity for water.
Hydrophobic. Weak affinity for water.
Lipophilic. Strong affinity for fats or other lipids.
Micro-droplet. A very small drop or minute droplet (as 0.1 to 0.01 of a drop). A drop is the smallest practical unit of liquid measure varying in size according to the specific gravity and viscosity of the liquid and to the conditions under which the drop is formed.
Miscible. Capable of mixing in any ratio without separation of the two phases. The mixture formed by a miscible liquid or solid can be a solution or dispersion (emulsion or suspension).
Miscible oil. Hydrocarbon oil with emulsifiers. Forms a milky emulsion in water. Oil soluble. Dissolves in oil.
Oil soluble skin beneficial ingredient. A skin beneficial ingredient that is oil-soluble. Examples include ascorbyl palmitate, tetrahexadecyl ascorbate, ascorbyl 2-glucoside, Vitamin A, Vitamin E, vitamin E acetate, Tocotrienol, vitamin D, vitamin K, Phytantriol, progesterone, testosterone, pregnenolone, dehydroepiandrosterone, androstenedione, estradiol, diosgenin, soy isoflavones, melatonin, benzocaine, benzyl alcohol, lidocaine, butamben, dibucaine, dimethisoquin, dyclonine, pramixine, tetracaine, triclosan, triclocarbanilide, bacitracin, chlortetracycline, neomycin, polymixin, tolnaftate, clioquinol, haloprogin, miconazole, povidone-iodine, undecylenic acid, benzophenone-3, ethylhexyl methoxycinnamate, homosalate, avobenzone, ethylhexyl salicylate, octocrylene, menthyl anthranilate, PABA, cinoxate, dioxybenzone, oxybenzone, padimate O, phenylbenzimidazole sulfonic acid, sulisobenzone, trolamine salicylate, benzoyl peroxide, calcium peroxide, sodium peroxide, urea peroxide and other such materials. These examples and other examples provided herein are given by way of illustration and not to limit the scope of compositions covered by a particular term.
Oil Soluble Vitamin C. Sodium ascorbyl palmitate.
Oil Soluble Vitamin E. Vitamin E and Vitamin E derivatives including, but not limited to, Vitamin E acetate.
Oleophilic. Strong affinity for oils.
Organic. Being, containing, or relating to carbon compounds, especially in which hydrogen is attached to carbon whether derived from living organisms or not.
Organic solvent. A solvent including a carbon compound. Examples include, without limitation, glycerin, PEG-6 (Polyethylene glycol 300), and Mpdiol glycol.
Rheology modifier. An ingredient that modifies the viscosity and flow of a composition. Examples include Aristoflex AVC (Ammonium Acryloyldimethyltaurate/VP Copolymer), Structure Plus and Structure XL (Acrylates/Aminoacrylates/c10-30 Alkyl PEG-20 Itaconate Copolymer), Carbomer, Xanthan Gum, Carbopol ETD 2020 (Acrylate C10-30 Alkyl Acrylate Crosspolymer), Rheocin (trihydroxystearin), Hydramol PGDS (PEG-90 Diisostearate), C24-28 Alkyl Dimethicone, and Behenyl alcohol.
Silicone. An organic siloxane, especially any of a large group of polymerized organic siloxanes that are unusually stable over a wide temperature range, that are obtained as oily fluids, resins and elastomers convertible into greases and other compounds, coatings, and rubbers, and that are used chiefly in waterproofing, lubrication, and electric insulation.
Silicone derivative. A silicone with one or more attributes beneficial to skin. Such attributes include emolliency, moisturization, skin smoothing, wrinkle reduction, skin softening, promoting normal skin growth or health, etc. Examples of silicone derivations, include, without limitation, most organosilicones, organic siloxanes, and their cross polymer (e.g., dimethicone, dimethicone copolyol, cetyl dimethicone copolymer, cetyl dimethicone, stearyl dimethicone, stearoxydimethicone, behenoxydimethicone, alkyl methicone, amodimethicone, dimethicone alkyl betaine, cyclomethicone, polydimethylsiloxane, diphenyldimethyl polysiloxane, silicone elastomers, cyclomethicone and dimethicone crosspolymer, Jeesilc 6056, Dow Corning 2501).
Skin beneficial composition. A composition with attributes beneficial to skin. Examples of such attributes include emolliency, moisturization, skin smoothing, wrinkle reduction, skin softening, and promoting normal growth or health. Examples of particular ingredients include oil-soluble skin beneficial ingredients; water-soluble skin beneficial ingredients; hydroquinone, arbutin, hydroquinone derivatives and other skin whitening agents; dimethylaminoethanol (DMEA), alpha-lipoic acid, coenzyme Q10 (ubiquinone), carnosine, glutathione, and other anti-wrinkle and anti-aging agents; vitamin C; vitamin E; water-soluble vitamin C derivatives, glycolic acid, lactic acid, mandelic acid, and hydroxy acid derivatives.
Solution. A solid, liquid, or gas mixed homogeneously with a liquid.
Solvent. A substance capable of or used in dissolving or dispersing one or more other substances, especially a liquid component of a solution present in greater amount than the solute.
Surfactant. In general, a surfactant is a substance useful for its cleansing, wetting, dispersing, or similar powers. Each of such functions is performed by a surfactant due to its ability to reduce surface tension. More specifically, as used herein, a surfactant is an ingredient that reduces the surface tension of water. Examples of surfactants include, without limitation, Tauranol I-78-6, Tauranol ws conc., Lathanol LAL, Dow Corning 2501, Hydramol PGDS.
Suspension. Particles mixed in a fluid or a solid, but undissolved.
Water miscible organic solvent. An organic solvent that can be mixed with water without separation of the water from the organic solvent. In the practice of the invention, the preferred (but not required) water miscible organic solvents are those commonly used in cosmetic applications, for example, glycerin, ethylene glycol, propylene glycol, butylene glycol, hexylene glycol, pyrrolidone, N-methyl pyrrolidone, dimethyl sulfoxide, dimethyl sulfone, polyethylene glycol, polypropylene glycol, methylpropanediol, and similar solvents.
Water soluble. Dissolves in water.
Water soluble skin beneficial ingredient. A skin beneficial ingredient that is water-soluble. Examples include hydroquinone, glycolic acid, lactic acid, mandelic acid, malic acid, salicylic acid, tartaric acid, aleuritic acid, arbutin, and other similar materials.
Water-soluble vitamin C. A water-soluble source of vitamin C. Examples include ascorbic acid, ascorbic acid derivatives (including, for example, sodium ascorbate, calcium ascorbate, magnesium ascorbate, sodium ascorbyl phosphate, magnesium ascorbyl phosphate, Ester C.
Vitamin C derivative. The combination in a composition of an oil-soluble form of vitamin C and a water-soluble form of vitamin C.

Vitamin C, also known as L-ascorbic acid, is water-soluble. Vitamin C is unstable in the presence of air when the vitamin C is in an aqueous medium. Therefore, vitamin C is unstable in water-based cosmetic formulations. Eliminating water from cosmetic formulations cures this problem. Aqueous cosmetic formulations in which vitamin C is stable can also be obtained by removing air from the formulations and by storing the formulations in an airtight container.

Hydroquinone is a water-soluble skin-whitening ingredient. Hydroquinone, like vitamin C, is also unstable when in a water-based formulation exposed to air. Further, when both vitamin C and hydroquinone are present in a water-based formulation, serious discoloration occurs when the formulation is exposed to air.

Current stable non-aqueous vitamin C products typically contain compositions such as waxes or combinations of waxes, oils, glycols, and siloxane elastomers. These compositions promote the stability of the vitamin C but leave an oily or waxy residue on skin after use. This residue is unappealing and unaesthetic.

Another solution to the instability of vitamin C in aqueous compositions is to use modified forms of vitamin C in aqueous formulations. Modified or derivative forms of vitamin C include ascorbic acid glucoside, sodium ascorbyl phosphate, magnesium ascorbyl phosphate, calcium ascorbyl phosphate, ester C, ascorbyl palmitate, and tetrahexyldecyl ascorbate. Formulations with modified forms of vitamin C have proven expensive, not equivalent in efficacy on an equal weight basis, or have encountered other problems that adversely affect the successful sale of such formulations. Modified forms of vitamin C, such as oil-soluble ascorbyl palmitate in combination with vitamin C can, however, be beneficial for certain topical applications.

Recent reports indicate that combining vitamin C with significant amounts of certain oil-soluble ingredients such as vitamin E and a sunscreen can provide synergistically enhanced skin protection. For example, a combination of vitamin C (10% by weight), vitamin E (2% by weight), and Benzophenone-3 (0.25% by weight) was found effective. Additional examples of synergistically beneficial effects of the combination of vitamins C and E have also been reported.

Consequently, it would be highly desirable to provide formulations that contain both water-soluble and oil soluble ingredients, and, in particular, to have such formulations in which the combination of water-soluble and oil-soluble ingredients provide known synergistic benefits. The formulation of compositions including water-soluble and oil-soluble ingredients is, however, difficult. One problem is that certain water-soluble vitamins like vitamin C are, as noted, inherently unstable in formulations that include any significant amount of water. Another problem is that vitamin C is not soluble in oil-soluble cosmetic ingredients. A further problem is that oil-soluble ingredients like vitamin E are not water-soluble. Still another problem is that the formulation of oil-soluble ingredients in a water-based formulation typically requires the use of an emulsifier. Several recent patents have addressed this problem. These patents include U.S. Pat. Nos. 6,235,272, 6,174,519, 6,036,946, and 4,704,280. Most of the preferred combinations of vitamins C and E in these patents permit only up to about 2.2% vitamin C and 0.5% vitamin E, even though the claims set forth up to 7.0% of vitamin C and up to 30.0% of vitamin E. None of the foregoing patents appear to claim more than 7.0% by weight vitamin C (a water soluble ingredient) in combination with any amount of vitamin E (an oil soluble ingredient). Brandt et al. (U.S. Pat. No. 6,696,067) discloses certain combinations of water soluble and oil soluble antioxidants, such as ascorbic acid, Vitamin E acetate, tocopherol, or ascorbyl palmitate, but only in amounts ranging from 0.01% to 5% by weight. The combinations over 5% are not possible by Brandt disclosure.

I have discovered an improved topical formulation that includes water-soluble and oil-soluble ingredients in typically much greater than 5% combinations. The formulation includes a water-soluble composition(s), an oil-soluble composition(s), water, a rheology modifier(s), and a water-miscible organic solvent(s). One advantage of the topical formulation of the invention is that it ordinarily does not impart a sticky or oily skin feel. In most cosmetic formulations, both vitamin C and vitamin E produce a sticky feel when applied in substantial amounts to skin.

Water is critical in the formulation of the invention because water dissolves at least a portion of water-soluble ingredients. The water—water-soluble ingredient solution is then acted on by the rheology modifier and the water miscible organic solvent to stabilize the water-soluble ingredient.

The rheology modifier is, in combination with the water, critical in the formulation of the invention because it functions to stabilize both the water-soluble composition and the oil-soluble composition. A portion of the water-soluble composition dissolves in the water. A portion of the water is absorbed by the rheology modifier. When the rheology modifier absorbs water it swells, i.e., the volume of the rheology modifier increases. The portion of water-soluble composition(s) that is in water absorbed by the rheology modifier is stabilized because the rheology modifier functions, practically speaking, to house and shield the water-soluble composition and to prevent it from dispersing from the rheology modifier. In addition, once a portion of the water is absorbed by the rheology modifier, the rheology modifier and oil-soluble composition form an emulsion. The formation of the emulsion further stabilizes the formulation because it makes it more difficult for the water-soluble composition to separate from the oil-soluble composition.

The water miscible organic solvent is also critical in the formulation of the invention because it solubilizes the water-soluble ingredient when the organic solvent mixes with the water. When the organic solvent and water mix, separation of the water-soluble composition and oil soluble composition is made yet more difficult. As noted below, the organic solvent also functions to stabilize the oil-soluble ingredient(s) in the formulation.

The amount of water-soluble composition in the formulation is from over 5% to up to 30.0% by weight, preferably from over 7% up to 30.0% by weight, and most preferably from over 10% up to 30% by weight. A particular advantage of the invention is that relatively large amounts of ascorbic acid, from over 5% to up to about 30% by weight can be combined with relatively large amounts of vitamin E or other oil-soluble components. If the amount of the water-soluble ingredient is in excess of 30%, the amount of water required becomes excessive. Consequently, quantities of ascorbic acid or other water-soluble compositions greater than about 30% are presently not practical in the practice of the invention. It is theorized that this is the case because as more water-soluble compositions are utilized, more water is required. And, the amount of water appears limited by the amount of water-miscible organic solvent required.

The amount of oil-soluble composition in the formulation is 0.05% to 30% by weight, preferably from 1.0% to 25.0% by weight, most preferably from 2% to 25% by weight. The amount of oil-soluble composition in the formulation can also be from 5% to 25% because one of the advantages of the invention is being able to incorporate unusually large amounts of an oil-soluble composition.

The amount of rheology modifier in the formulation is from 0.05% to 10% by weight, preferably from 0.5% to 5.0% by weight.

The amount of water in the formulation is from 0.05% to 25%, preferably 1.0% to 15% by weight.

The amount of water-miscible organic solvent in the formulation is from 5% to 82%, preferably 7.5 to 82% by weight, and most preferably 10.0% to 80.0% by weight. The use of a water miscible organic solvent is believed critical in the practice of the invention because it facilitates the intermixing of water—which contains at least a portion of the water-soluble composition—with the organic solvent. Further, the organic solvent functions to help the suspension of oil-soluble ingredients as micro-droplets. These micro-droplets do not coalesce to make bigger droplets in the present of the organic solvent. In addition, when water mixes with the organic solvent, each molecule of water occupies an empty space among the organic solvent molecules, and the organic solvent molecules function to interfere with the coalescing of the water into larger droplets.

The amount of water in the formulation is from 0.05% to 25%, preferably 1% to 15%. As earlier discussed, in many cosmetic formulations water is excluded to increase the stability of the ascorbic acid and decrease the likelihood that oil-soluble ingredients will separate from the formulation. In contrast, in the formulation of the invention, water is desirable and required in the formulation for reasons set forth above.

The proportion of water to water-miscible organic solvent in the formulation appears to be important in terms of vitamin C stability and is in the range of 1:2 to 1:5, preferably 1:3 to 1:4. This proportion is believed important (although this belief has not presently been proven) because there must be sufficient water-miscible organic solvent to absorb and/or mix with water containing vitamin C to stabilize the vitamin C and to facilitate the suspension of micro-droplets of oil soluble ingredients. The rheology modifier works hand-in-hand with the organic solvent because the rheology modifier absorbs some of the water in the formulation.

Water used in the formulations of the invention can be provided by direct addition of water, can be provided from other ingredients in the formulation that contain water in their own composition, or can consist of water from hydrated ingredients.

Other water soluble or water dispersible ingredients that can be used in the formulation of the invention include, by way of example and not limitation, other vitamins, anti-inflammatory agents, anesthetics, analgesics, enzymes, UV-absorbers, antiperspirants, deodorants, colorants, hydroxy aids, skin lightening agents, emollients, medications, antibiotics, antifungal agents, and insect repellents. If desired, such ingredients need not be water-soluble or water dispersible. Such ingredients can, if desired, be oil-soluble.

The following examples are presented by way of illustration, and not limitation, of the invention. In each example, the amount or proportion of each ingredient is in weight percent unless otherwise indicated.

### EXAMPLE I

The following ingredients are provided.

| Ingredient | Weight Percent |
|---|---|
| PEG-6 (polyethylene glycol) | 60.65 |
| Aristoflex AVC (Rheology modifier) | 1.00 |
| Deionized water | 15.00 |
| Ascorbic acid (water soluble ingredient) | 10.50 |
| Vitamin E acetate (oil soluble ingredient) | 2.00 |
| Benzophenone-3 (oil soluble ingredient) | 0.25 |
| Killitol (preservative) | 0.30 |
| Jeesilc 6056 (Dimethyl Polysiloxane) | 10.00 |
| (oil soluble ingredient) | |
| Actiplex 2790 (botanical blend) | 0.10 |
| Titanium dioxide | 0.20 |

The ascorbic acid is added to the water. The remaining ingredients are mixed separately and are then added to the aqueous ascorbic acid solution to produce a facial and body cream. The cream is put in a container.

### EXAMPLE II

The following ingredients are provided.

| Ingredient | Weight Percent |
|---|---|
| PEG-6 | 61.30 |
| Aristoflex AVC (Rheology modifier) | 0.80 |
| Deionized water | 15.00 |
| Ascorbic acid (water soluble ingredient) | 10.50 |
| Vitamin E acetate (oil soluble ingredient) | 2.00 |
| Killitol (preservative) (chlorphenesin, methyl paraben) | 0.30 |
| Jeechem HPIB (a blend of silicones) | 10.00 |
| (oil soluble ingredient) | |
| Actiplex 2790 (botanical extracts including flavanoids) | 0.10 |

All of the ingredients are mixed. The mixture is heated and stirred at 60 to 70 degrees C. until the mixture is homogenous, i.e., for about five to ten minutes. The homogeneous mixture is cooled to room temperature. A skin cream is produced. The cream is put in a container. This example demonstrates the use of an oil-soluble silicone derivative (Jeechem HPIB, which is a blend of polydimethylsiloxane, cyclized dimethicone, and cyclomethicone) in combination with ascorbic acid and vitamin E acetate.

### EXAMPLE III

The following ingredients are provided.

| Ingredient | Weight Percent |
|---|---|
| PEG-6 | 63.70 |
| Aristoflex AVC (Rheology modifier) | 0.80 |
| Deionized water | 15.00 |
| Sodium Ascorbyl Phosphate (water soluble ingredient) | 10.00 |
| Killitol | 0.30 |
| Jeesilc 6056 (oil soluble ingredient) | 10.00 |
| Titanium dioxide | 0.20 |

All of the ingredients are mixed. The mixture is heated and stirred at 60 to 70 degrees C. until the mixture is homogenous, i.e., for about five to ten minutes. The homogeneous mixture is cooled to room temperature. A skin cream is produced. The cream is put in a container. This example demonstrates the use of an oil-soluble form of vitamin C.

### EXAMPLE IV

The following ingredients are provided.

| Ingredient | Weight Percent |
|---|---|
| PEG-6 | 65.8 |
| Aristoflex AVC (Rheology modifier) | 0.80 |
| Deionized water | 15.00 |
| Ascorbic acid (water soluble ingredient) | 4.00 |
| Hydroquinone USP (water soluble ingredient) | 4.00 |
| Killitol | 0.30 |
| Jeechem HPIB (oil soluble ingredient) | 10.00 |
| Lactic acid (water soluble ingredient) | 0.10 |

All of the ingredients are mixed. The mixture is heated and stirred at 60 to 70 degrees C. until the mixture is homogenous, i.e., for about five to ten minutes. The homogeneous mixture is cooled to room temperature. A skin lotion is produced. The cream is put in a container. This example demonstrates the preparation of a skin whitening formula that contains ascorbic acid, lactic acid and hydroquinone with water. Both ascorbic acid and hydroquinone are known in prior art to easily oxidize when water and air are both present.

### EXAMPLE V

The following ingredients are provided.

| Ingredient | Weight Percent |
|---|---|
| PEG-6 | 59.7 |
| Aristoflex AVC (Rheology modifier) | 1.00 |
| Deionized water | 15.00 |
| Niacinamide Ascorbate (water soluble ingredient) | 10.00 |
| Hydroquinone USP (water soluble ingredient) | 4.00 |
| Killitol | 0.30 |
| Cetyl Dimethicone Copolyol (oil soluble ingredient) | 5.00 |
| Cyclomethicone and Dimethiconol (oil soluble | 5.00 |
| ingredient) | |

All of the ingredients are mixed. The mixture is heated and stirred at 60 to 70 degrees C. until the mixture is homogenous, i.e., for about five to ten minutes. The homogeneous mixture is cooled to room temperature. A skin lotion is produced. The cream is put in a container. This example demonstrates the use of ascorbyl palmitate in a silicone surfactant base. The amount of hydroquinone and/or hydroquinone derivative in a composition of the invention is in the range of 0.1% to 10% by weight, preferably 1% to 4% by weight. The amount of sodium ascorbyl palmitate in a composition of the invention is in the range of 0.01% to 10.0%.

### EXAMPLE VI

The cream of Example I is stored at room temperature in a container. A lid is laid across the top of the container to slow evaporation. The lid does not prevent ambient air from slowly entering the container. After six months the color of the cream has not changed.

### EXAMPLE VII

The cream of Example I is stored at room temperature in a container. A lid is laid across the top of the container to slow evaporation. The lid does not prevent ambient air from slowly entering the container. After one year the color of the cream has changed slightly and is somewhat darker.

### EXAMPLE VIII

Examples VI and VII are repeated, except the cream of Example II is utilized instead of the cream of Example I. Similar results are obtained.

### EXAMPLE IX

Examples VI and VII are repeated, except the lotion of Example III is utilized instead of the cream of Example I. Similar results are obtained.

### EXAMPLE X

Examples VI and VII are repeated except that the lotion of Example IV is utilized instead of the cream of Example I. Similar results are obtained.

### EXAMPLE XI

Examples VI and VII are repeated except that the cream of Example V is utilized instead of the cream of Example 1. Similar results are obtained.

### EXAMPLE XII

The following ingredients are provided.

| Ingredient | Weight percent |
|---|---|
| Methylpropanediol | 65.5 |
| Aristoflex AVC (Rheology modifier) | 0.8 |
| Titanium Dioxide | 0.2 |
| Deionized Water | 15.0 |
| Ascorbic acid (water soluble ingredient) | 2.0 |
| Hydroquinone (water soluble ingredient) | 4.0 |
| Jeesilc 6056 (oil soluble ingredient) | 10.0 |
| Killitol | 0.5 |
| Lactic Acid (water soluble ingredient) | 2.0 |

A composition is prepared using the process used to prepare to lotion of Example V. A liquid serum is obtained.

### EXAMPLE XIII

The following ingredients are provided.

| Ingredient | Weight percent |
|---|---|
| Deionized Water | 15.0 |
| Aristoflex AVC (Rheology modifier) | 1.0 |
| Peg-6 | 62.6 |
| Ascorbic Acid (water soluble ingredient) | 6.0 |
| Lactic Acid (water soluble ingredient) | 2.0 |
| Lipoic Acid | 0.5 |
| Sodium Hyaluronate | 0.1 |
| Dimethylaminoethanol (DMEA) | 0.1 |
| Vitamin E Acetate (oil soluble ingredient) | 2.0 |
| Killitol | 0.5 |
| Jeesilc 6056 (water soluble ingredient) | 10.0 |
| Titanium Dioxide | 0.2 |

A composition is prepared using the process used to prepare the lotion of Example V. A white, liquid lotion is obtained.

### EXAMPLE XIV

This example shows the formulation of Benzocaine, a water-insoluble topical analgesic ingredient with glycerin, a water-soluble ingredient.

| Ingredient | Weight Percent |
|---|---|
| 1. PEG-6 | 49.8 |
| 2. Aristoflex AVC (Rheology modifier) | 0.5 |
| 3. Glycerin | 5.0 |
| 4. Deionized Water | 20.0 |
| 5. Vitamin E Acetate (oil soluble ingredient) | 0.1 |
| 6. Geogard 221 (Preservative, including | 0.5 |
| dehydroacetic acid and benzyl | |
| alcohol) | |
| 7. Jeechem HPIB (oil soluble ingredient) | 10.0 |
| 8. Actiplex (Plant Extracts Blend, | 0.1 |
| including flavanoids) | |
| 9. Benzocaine (oil soluble ingredient) | 4.0 |
| 10. PEG-6 | 10.0 |

Mix ingredients 2, 3 and 4 together until a clear gel is formed. Add all other ingredients, except 9 and 10, to the gel and mix to form the main batch. Mix ingredients 9 and 10 together and heat at 40 to 50 degrees C. to form a clear solution. Add this solution to the main batch with mixing and cool to room temperature. A clear cream is obtained. The cream contains a solubilized form of benzocaine that is highly bioavailable.

### EXAMPLE XV

This example shows the formulation of Progesterone, an oil-soluble hormone.

| Ingredient | Weight Percent |
|---|---|
| 1. Peg-6 | 44.8 |
| 2. Aristoflex AVC (Rheology modifier) | 0.5 |
| 3. Glycerin (water soluble ingredient) | 10.0 |
| 4. Deionized Water | 20.0 |
| 5. Vitamin E Acetate (oil soluble ingredient) | 0.1 |
| 6. Geogard 221 (Preservative) | 0.5 |
| 7. Jeechem HPIB (oil soluble ingredient) | 10.0 |
| 8. Actiplex (Plant Extracts Blend) | 0.1 |
| 9. Progesterone (oil soluble ingredient | 4.0 |
| 10. PEG-6 | 10.0 |

Mix ingredients 2, 3 and 4 together until a clear gel is formed. Add all other ingredients, except 9 and 10, to the clear gel and mix to produce a main batch. Mix ingredients 9 and 10 together and heat at 40 to 50 degrees C. to form a clear solution. Add this solution to the main batch with mixing and cool to room temperature. A clear cream is obtained. The cream contains solubilized form of progesterone that is highly bioavailable.

### EXAMPLE XVI

This example shows the formulation of both water-soluble and oil-soluble sunscreen ingredients in the same formula.

| Ingredient | Weight Percent |
|---|---|
| 1. PEG-6 | 34.3 |
| 2. Aristoflex AVC (Rheology modifier) | 0.5 |
| 3. Glycerin (water soluble ingredient) | 5.0 |
| 4. Deionized Water | 20.0 |
| 5. Vitamin E Acetate | 0.1 |
| 6. Geogard 221 (Preservative) | 0.5 |
| 7. Jeechem HPIB (oil soluble ingredient) | 10.0 |
| 8. Actiplex (Plant Extracts Blend) | 0.1 |
| 9. Benzophenone-3 (oil soluble ingredient) | 4.0 |
| 10. Avobenzone (oil soluble ingredient) | 3.0 |
| 11. Ethylhexyl Methoxycinnamate (oil soluble | 7.5 |
| ingredient) | |
| 12. Homosalate (oil soluble ingredient) | 5.0 |
| 13. PEG-6 | 10.0 |

Mix ingredients 2, 3 and 4 together until a clear gel is formed. Add all other ingredients, except 9, 10, 11, 12, and 13, to the clear gel and mix to form a main batch. Mix ingredients 9, 10, 11, 12 and 13 together and heat at 40 to 50 degrees C. to form a clear solution. Add this clear solution to the main batch with mixing and cool to room temperature. A clear, light yellow lotion is obtained. This contains solubilized form of sunscreen that is very cosmetically appealing and very effective at blocking UV light.

### EXAMPLE XVII

This example shows the formulation of oil-soluble and water-soluble botanical cosmeceuticals for body slimming and muscle-firming benefits.

| | Weight |
|---|---|
| Ingredients | Percent |
| 1. PEG-6 | 0.9 |
| 2. Aristoflex AVC (Rheology modifier) | 0.8 |
| 3. Glycerin (Water-soluble ingredient) | 5.0 |
| 4. Deionized Water | 50.0 |
| 5. Vitamin E Acetate | 0.1 |
| 6. Geogard 221 (Preservative) | 0.5 |
| 7. Jeechem HPIB (oil soluble ingredient) | 2.0 |
| 8. Forskohlin (oil soluble ingredient) | 0.1 |
| 9. Pleurimincyl (water soluble ingredient) | 1.0 |
| 10. L-Carnitine (water soluble ingredient) | 0.5 |
| 11. Aminophylline (water soluble ingredient) | 0.5 |
| 12. PEG-6 (solubilizer) | 8.5 |
| 13. Actiplex (Plant Extracts Blend) | 0.1 |

Mix ingredients 2, 3, and 4 together until a clear gel is formed. Add ingredients 1, 5, 6, 7, 10, 11, and 13 to the clear gel and mix to form a main batch. Mix ingredients 8, 9, and 12 together and heat at 40 to 50 degrees C. to form a solution. Add the solution to the main batch and mix. Cool to room temperature. A translucent cream is obtained.

### EXAMPLE XVIII

This example shows the formulation of Aloe Vera, a water-soluble skin care ingredient, and Progesterone, an oil-soluble skin care and medicinal ingredient. Additional oil-soluble skin-smoothing ingredients, such as several silicone derivatives and Vitamin E, are also included.

| Ingredient | Weight Percent |
|---|---|
| 1. PEG-6 | 44.1 |
| 2. Aristoflex AVC (Rheology modifier) | 1.0 |
| 3. Glycerin (water soluble ingredient) | 5.0 |
| 4. Aloe Vera (water soluble ingredient) | 0.3 |
| 5. Deionized Water | 15.0 |
| 6. Vitamin E Acetate (oil soluble ingredient) | 0.1 |
| 7. Geogard 221 (Preservative) | 0.5 |
| 8. Dimethicone (oil soluble ingredient) | 4.0 |
| 9. Cyclomethicone (oil soluble ingredient) | 4.0 |
| 10. Dimethiconol (water soluble ingredient) | 2.0 |
| 11. Cetyl Dimethicone Copolyol | 2.0 |
| (oil soluble ingredient) | |
| 12. PEG-6 | 20.0 |
| 13. Progesterone (oil soluble ingredient) | 2.0 |

Mix ingredients 2, 3, 4, and 5 together until a clear gel is formed. Add ingredients 1, 6, 7, 8, 9, 10, and 11 to the clear gel and mix to form a main batch. Mix ingredients 12 and 13 together and heat at 50 to 60 degrees C. to form a solution. Add the solution to the main batch and mix. A translucent cream is obtained. The cream is rapidly absorbed by the skin on application.

### EXAMPLE XIX

This example shows in-situ preparation of Nicotinamide Ascorbate, a water-soluble ingredient, and its formulation with oil-soluble vitamin E acetate.

| Ingredient | Weight Percent |
|---|---|
| 1. PEG-6 | 46.4 |
| 2. Aristoflex AVC (Rheology modifier) | 1.0 |
| 3. Glycerin (water soluble ingredient) | 5.0 |
| 4. Deionized Water | 20.0 |
| 5. itamin E Acetate (oil soluble ingredient) | 2.1 |
| 6. Geogard 221 (Preservative) | 0.5 |
| 7. Dimethicone (water soluble ingredient) | 4.0 |
| 8. Nicotinamide (water soluble ingredient) | 6.0 |
| 9. Ascorbic Acid (water soluble ingredient) | 9.0 |
| 10. Dimethiconol (water soluble ingredient) | 4.0 |
| 11. Cetyl Dimethicone Copolyol (oil soluble ingredient) | 2.0 |

Mix ingredients 2, 3, and 4 together until a clear gel is formed. Add ingredients 1, 5, 6, 7, 8, 9, 10, and 11 to the clear gel and mix. A translucent cream is obtained. The cream is rapidly absorbed by skin on application.

### EXAMPLE XX

A liquid serum product with water-soluble ingredients, ascorbic acid and Glucosamine, and oil-soluble ingredients, Vitamin A and Vitamin E.

| Ingredients | Weight Percent |
|---|---|
| Glucosamine HCl (water soluble ingredient) | 6.0 |
| Deionized Water | 15.0 |
| Caustic Soda | qs |
| Ascorbic Acid (water soluble ingredient) | 5.0 |
| Mandelic Acid (water soluble ingredient | 1.0 |
| Lactic Acid (water soluble ingredient) | 0.5 |
| Aloe Vera (water soluble ingredient) | 1.0 |
| Glycerin | 56.3 |
| Dow Corning 2501 (water soluble ingredient) | 10.0 |
| Structure Plus (Rheology modifier) | 4.0 |
| Actiphyte of Eyebright | 0.1 |
| Actiplex 2790 | 0.1 |
| Vitamin A Palmitate (oil soluble ingredient) | 0.5 |
| Vitamin E Acetate (oil soluble ingredient) | 0.5 |

Glucosamine HCl is dissolved in water to form a solution. The solution is neutralized with caustic soda to pH 6.5. All other ingredients are added to this neutralized solution and the resulting composition is mixed at 40 to 50 degrees C. for 30 minutes. The composition is cooled to room temperature. A gel-like product is obtained.

### EXAMPLE XXI

This example shows the formulation of a complex mixture of water-soluble and oil-soluble compositions.

| Ingredient | Weight Percent |
|---|---|
| PEG-6 | 43.2 |
| Aristoflex AVC (Rheology modifier) | 1.0 |
| Glycerin (water soluble ingredient) | 5.0 |
| Deionized Water | 15.0 |
| Activera (water soluble ingredient) | 0.3 |
| Geogard 221 | 0.5 |
| L-Carnitine (water soluble ingredient) | 0.5 |
| PEG-6 | 20.0 |
| Vitamin E Acetate (oil soluble ingredient) | 0.1 |
| Centella Asiatica (oil soluble ingredient) | 0.5 |
| Tetrahydrocurcumin (oil soluble ingredient) | 0.2 |
| Escin (oil soluble ingredient) | 0.5 |
| Esculin (oil soluble ingredient) | 0.5 |
| Boswellia Serrata Extract (oil soluble ingredient) | 0.5 |
| Dimethicone (oil soluble ingredient) | 4.0 |
| Dow 1401 Fluid (oil soluble ingredient) | 2.0 |
| Abil EM 90 (oil soluble ingredient) | 2.0 |
| Titanium Dioxide | 0.2 |
| Jeesilc 6056 (oil soluble ingredient) | 4.0 |

The Aristoflex, glycerin, and deionized water are mixed together to form a clear gel. The Vitamin E Acetate, Centella Asiatica, Tetrahydrocurcumin, Escin, Esculine, Boswellia Serata and PEG (20% portion) are mixed together to form a solution. This solution is mixed together with the gel to form a primary mixture, then the other remaining ingredients are added to the primary mixture to form a final composition. The final composition is heated to a temperature in the range of 50 degrees C. to 60 degrees C. and is mixed. The final composition is then cooled to room temperature to form a cream.

1. A method of forming a topical formulation including water-soluble ingredients and oil-soluble ingredients stabilized in said topical formulation, said method comprising the steps of
(a) providing at least one water-soluble composition;
(b) providing at least one oil-soluble composition;
(c) providing water;
(d) providing at least one water miscible organic solvent that admixes with water such that molecules of water occupy empty spaces among molecules of the organic solvent;
(e) providing at least one rheology modifier to
(i) absorb at least a portion of said water and of said water soluble composition dissolved in said water, and,
(ii) form an emulsion with said oil-soluble composition;
(f) admixing said water-soluble composition, said oil-soluble composition, said water, said water miscible organic solvent, and said rheology modifier such that
(i) at least a portion of said water-soluble composition is dissolved by said water,
(ii) at least a portion of said water is absorbed by said rheology modifier,
(iii) said rheology modifier, including any of said water absorbed by said modifier, forms an emulsion with said oil-soluble composition, and
(iv) at least a portion of said water mixes with said organic solvent such that molecules of said water occupy empty space among molecules of said organic solvent.

2. A topical formulation with water-soluble and oil-soluble ingredients, said formulation comprising
(a) From over 5.0% to up to 30% by weight of at least one water-soluble composition;
(b) 1.0% to 25% by weight of at least one oil-soluble composition;
(c) 0.05% to 25% by weight water;
(d) 0.05% to 10% by weight of at least one rheology modifier; and,
(e) 7.5% to 82% by weight of at least one water miscible organic solvent.

3. A topical formulation with water-soluble and oil-soluble ingredients, said formulation comprising
(a) From over 5.0% to up to 30% by weight of water-soluble vitamin C;
(b) 1.0% to 25% by weight of oil-soluble Vitamin E;
(c) 0.05% to 25% by weight water;
(d) 0.05% to 10% by weight of at least one rheology modifier; and,
(e) 5.0% to 82% by weight of at least one water miscible organic solvent.

4. The formulation of claim 2 including 0.1% to 10.0% by weight of a skin beneficial composition selected from a group consisting of hydroquinone and hydroquinone derivatives.

5. The formulation of claim 2 including 0.01% to 10.0% by weight of oil soluble vitamin C consisting of sodium ascorbyl palmitate.

6. The formulation of claim 2 including
(f) 0.1% to 10.0% by weight of a skin beneficial composition selected from a group consisting of hydroquinone and hydroquinone derivatives; and
(g) 0.01% to 10.% by weight of oil soluble vitamin C consisting of sodium ascorbyl palmitate.

7. The formulation of claim 3 including 0.1% to 10.0% by weight of a skin beneficial composition selected from a group consisting of hydroquinone and hydroquinone derivatives.

8. The formulation of claim 3 including 0.01% to 10.0% by weight of oil soluble vitamin C consisting of sodium ascorbyl palmitate.

9. The formulation of claim 3 including
(a) 0.1% to 10.0% by weight of a skin beneficial composition selected from a group consisting of hydroquinone and hydroquinone derivatives; and
(b) 0.01% to 10.% by weight of oil soluble vitamin C consisting of sodium ascorbyl palmitate.

10. The formulation of claim 1 including 0.1% to 10.0% by weight of a skin beneficial composition selected from a group consisting of hydroquinone and hydroquinone derivatives.

11. The formulation of claim 1 including 0.01% to 10.0% by weight of oil soluble vitamin C consisting of sodium ascorbyl palmitate.

12. The formulation of claim 1 including
(h) 0.1% to 10.0% by weight of a skin beneficial composition selected from a group consisting of hydroquinone and hydroquinone derivatives; and
(i) 0.01% to 10.% by weight of oil-soluble vitamin C consisting of sodium ascorbyl palmitate.

13. The formulation of claim 1 wherein said water-soluble composition comprises from over 7.0% up to 30% by weight of water-soluble vitamin C.

14. The formulation of claim 1 wherein said oil-soluble composition comprises from 1.0% to 25% by weight of oil-soluble vitamin E.

15. The formulation of claim 1 wherein
(a) said water-soluble composition comprises from over 5.0% up to 30% by weight of water-soluble vitamin C, and
(b) said oil-soluble composition comprises from 1.0% to 25% by weight of oil-soluble vitamin E.

16. The formulation of claim 1
(a) wherein said water-soluble composition comprises from over 5.0% up to 30% by weight of water-soluble vitamin C;
(b) wherein said oil-soluble composition comprises from 1.0% to 25% by weight of oil-soluble vitamin E; and,
(c) including from 1.0% to 25% by weight of a skin beneficial composition selected from a group consisting of hydroquinone and hydroquinone derivatives.

17. The formulation of claim 16 including 0.01% to 10.0% by weight of oil-soluble vitamin C consisting of sodium ascorbyl palmitate.

18. The topical formulation of claim 3 wherein the amount of water-soluble vitamin C is from over 7.0% up to 30% by weight.
